(19) United States
(12) Patent Application Publication
(10) Pub. No.: US 2019/0008848 A1
(43) Pub. Date: Jan. 10, 2019

(54) IDENTIFICATION OF ADDITIONAL ANTI-PERSISTER ACTIVITY AGAINST BORRELIA BURGDORFERI FROM AN FDA DRUG LIBRARY

(71) Applicant: THE JOHNS HOPKINS UNIVERSITY, BALTIMORE, MD (US)

(72) Inventors: YING ZHANG, ELLICOTT CITY, MD (US); JIE FENG, BALTIMORE, MD (US)

(21) Appl. No.: 15/754,191

(22) PCT Filed: Aug. 19, 2016
(86) PCT No.: PCT/US2016/047784

§ 371 Date: Feb. 21, 2018

Related U.S. Application Data
(60) Provisional application No. 62/208,232, filed on Aug. 21, 2015.

(51) Int. Cl. A61K 31/485; A61P 31/04; A61K 31/409; A61K 31/4196; A61K 31/427; A61K 31/43; A61K 31/497; A61K 45/06; A61K 31/551; A61K 38/12

(52) U.S. Cl. CPC A61K 31/485; A61P 31/04; A61K 31/409; A61K 31/4196; A61K 38/12; A61K 31/43; A61K 31/497; A61K 45/06; A61K 31/551; A61K 31/427

(57) ABSTRACT

The presently disclosed subject matter provides methods for inhibiting the growth and/or survival of bacteria from the Borrelia genus and for treating Lyme disease. These methods include agents that inhibit the glutathione/γ-glutamyl pathway and are involved in protection against free radical damage, cell membranes, energy production, and/or reactive oxygen species production. Many of these compounds are antimicrobial agents (antibiotics, antivirals, antifungals, anthelmintics or antiparasitics) and drugs used for treating other non-infectious conditions.

### CROSS-REFERENCE TO RELATED APPLICATIONS

This application claims the benefit of U.S. Provisional Application No. 62/208,232, filed Aug. 21, 2015, which is incorporated herein by reference in its entirety.

### BACKGROUND

Lyme disease is a leading vector borne disease in the US. Although the majority of Lyme patients can be cured with standard 2-4 week antibiotic treatment, 10-20% of patients continue to suffer from prolonged post-treatment Lyme disease syndrome (PTLDS). While the cause for this is unclear, persisting organisms not killed by current Lyme antibiotics may be involved.

### SUMMARY

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of Dec. 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml.

The presently disclosed subject matter provides an additional 113 hits that have higher or equivalent activity against Borrelia persisters than the current antibiotics for Lyme disease. Many of these compounds are antimicrobial agents (antibiotics, antivirals, antifungals, anthelmintics or antiparasitics) used for treating other infections. These include antibacterials such as rifamycins (3-formal-rifamycin, rifaximin, rifamycin SV), thiostrepton, quinolone drugs (sarafloxacin, clinafloxacin, tosufloxacin), and cell wall inhibitors carbenicillin, tazobactam, aztreonam; antifungal agents such as fluconazole, mepartricin, bifonazole, climbazole, oxiconazole, nystatin; antiviral agents zanamivir, nevirapine, tilorone; antimalarial agents artemisinin, methylene blue, and quidaldine blue; antihelmintic and antiparasitic agents toltrazuril, tartar emetic, potassium antimonyl tartrate trihydrate, oxantel, closantel, hycanthone, pyrimethamine, and tetramisole. Drugs used for treating other non-infectious conditions including verteporfin, oltipraz, pyroglutamic acid (also known as pidolic acid), and dextrorphan tartrate, that act on glutathione/γ-glutamyl pathway involved in protection against free radical damage, and also antidepressant drug indatraline, were found to have high activity against stationary phase B. burgdorferi. Among the active hits, agents that affect cell membranes, energy production, and reactive oxygen species production are more active against the B. burgdorferi persisters than the commonly used Lyme antibiotics that inhibit macromolecule biosynthesis.

In an aspect, the presently disclosed subject matter provides a method for inhibiting the growth and/or survival of bacteria from the Borrelia genus, the method comprising contacting bacteria from the Borrelia genus with an effective amount of at least one agent that inhibits the glutathione/γ-glutamyl pathway.

In an aspect, the presently disclosed subject matter provides a method for treating Lyme disease in a patient in need thereof, the method comprising administering to a patient an effective amount of at least one agent that inhibits the glutathione/γ-glutamyl pathway in the patient.

In an aspect, the presently disclosed subject matter provides a method for inhibiting the growth and/or survival of bacteria from the Borrelia genus, the method comprising contacting bacteria from the Borrelia genus with an effective amount of at least one agent selected from the group consisting of verteporfin, thonzonium bromide, tetrachloroethylene, benzododecinium chloride, butyl chloride (1-chlorobutane), 3-formyl rifamycin, potassium antimonyl tartrate (tartar emetic), toltrazuril, thiostrepton, pyroglutamic acid, mepartricin, tilorone, oxantel, hycanthone, pyrimethamine, trilocarban (3,4,4′-trichlorocarbanilide), carbenicillin, oltipraz, bitoscanate, sarafloxacin, bacitracin, dextrorphan tartrate, tetramisole, bifonazole, ethacridine lactate, zanamivir, aluminum lactate, p-arsanilic acid, nifursol, nevirapine, rifaximin, oxibendazole, metrifonate, indatraline, florfenicol, benznidazole, ganciclovir, tazobactam, oxfendazole, phenothiazine, flubendazole, midecamycin, fluconazole, docosanol, aztreonam, benzoylpas (4-Benzamido salicylic acid), trifluridine, undecylenic acid, closantel, cefixime, thiamphenicol, ricobendazole (albendazole oxide), sulfamoxole, clopidol, tosufloxacin, metampicillin, amikacin, lamivudine, cephalosporin C, sulfachlorpyridazine, lomofungin, artesunate, valacyclovir, carzenide (4-carboxybenzenesulfonamide), clinafloxacin, efavirenz, cefsulodin, cloxyquin (5-chloro-8-hydroxy-quinoline), symclosene (trichloroisocyanuric acid), didanosine (2′-3′-dideoxyinosine), floxuridine (5-fluorodeoxyuridine), cyacetacide, roxithromycin, oxiconazole nitrate, climbazole, protionamide, ribavirin, griseofulvin, rifamycin SV, salicylanilide, diclazuril, imiquimod, penciclovir, nystatin, ampicillin, puromycin, stavudine (2′,3′-didehydro-3′-deoxythymidine), potassium iodide, voriconazole, penimepicycline, amantadine, nitroxoline (8-hydroxy 5-nitroquinoline), 4-aminosalicylic acid, ciclopirox olamine, nelfinavir mesylate, anisomycin, betamipron (n-benzoyl-b-alanine), famciclovir, flucytosine (5-fluorocytosine), clotrimazole, rimantadine, pazufloxacin, carbadox, amantadine, dibekacin, clorsulon, thiacetazone (amithiozone), fleroxacin, clofoctol, butoconazole nitrate, quinaldine blue, and methylene blue.

In an aspect, the presently disclosed subject matter provides a method for treating Lyme disease in a patient in need thereof, the method comprising administering to a patient an effective amount of at least one agent selected from the group consisting of verteporfin, thonzonium bromide, tetrachloroethylene, benzododecinium chloride, butyl chloride (1-chlorobutane), 3-formyl rifamycin, potassium antimonyl tartrate (tartar emetic), toltrazuril, thiostrepton, pyroglutamic acid, mepartricin, tilorone, oxantel, hycanthone, pyrimethamine, trilocarban (3,4,4′-trichlorocarbanilide), carbenicillin, oltipraz, bitoscanate, sarafloxacin, bacitracin, dextrorphan tartrate, tetramisole, bifonazole, ethacridine lactate, zanamivir, aluminum lactate, p-arsanilic acid, nifursol, nevirapine, rifaximin, oxibendazole, metrifonate, indatraline, florfenicol, benznidazole, ganciclovir, tazobactam, oxfendazole, phenothiazine, flubendazole, midecamycin, fluconazole, docosanol, aztreonam, benzoylpas (4-Benzamido salicylic acid), trifluridine, undecylenic acid, closantel, cefixime, thiamphenicol, ricobendazole (albendazole oxide), sulfamoxole, clopidol, tosufloxacin, metampicillin, amikacin, lamivudine, cephalosporin C, sulfachlorpyridazine, lomofungin, artesunate, valacyclovir, carzenide (4-carboxybenzenesulfonamide), clinafloxacin, efavirenz, cefsulodin, cloxyquin (5-chloro-8-hydroxy-quinoline), symclosene (trichloroisocyanuric acid), didanosine (2′-3′-dideoxyinosine), floxuridine (5-fluorodeoxyuridine), cyacetacide, roxithromycin, oxiconazole nitrate, climbazole, protionamide, ribavirin, griseofulvin, rifamycin SV, salicylanilide, diclazuril, imiquimod, penciclovir, nystatin, ampicillin, puromycin, stavudine (2′,3′-didehydro-3′-deoxythymidine), potassium iodide, voriconazole, penimepicycline, amantadine, nitroxoline (8-hydroxy 5-nitroquinoline), 4-aminosalicylic acid, ciclopirox olamine, nelfinavir mesylate, anisomycin, betamipron (n-benzoyl-b-alanine), famciclovir, flucytosine (5-fluorocytosine), clotrimazole, rimantadine, pazufloxacin, carbadox, amantadine, dibekacin, clorsulon, thiacetazone (amithiozone), fleroxacin, clofoctol, butoconazole nitrate, quinaldine blue, and methylene blue.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows images of B. burgdorferi culture (7 day old) incubated for 7 days with the indicated drugs, stained by SYBR green/PI assay, and examined using epifluorescence microscopy. Live cells are indicated by green fluorescence and dead cells are indicated by red fluorescence.

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml.

The presently disclosed subject matter relates to methods for inhibiting the growth and/or survival of bacteria from the Borrelia genus and for treating Lyme disease in a patient. Agents are disclosed that can inhibit growth of Borrelia bacteria and can be used for treating Lyme disease. These methods include agents that inhibit the glutathione/γ-glutamyl pathway.

I Methods for Inhibiting the Growth and/or Survival of Bacteria from the Borrelia Genus

In some embodiments, the presently disclosed subject matter provides a method for inhibiting the growth and/or survival of bacteria from the Borrelia genus, the method comprising contacting bacteria from the Borrelia genus with an effective amount of at least one agent that inhibits the glutathione/γ-glutamyl pathway.

The glutathione/γ-glutamyl pathway in mammalian cells is involved in protection against intracellular damage from free radicals and peroxides. Glutathione (GSH) is a reducing agent produced in the cytoplasm and transferred to the mitochondria by glutathione-s-transferase (GST), where it protects the mitochondria from ROS damage and functions in amino acid transport (Chiou et al., 2010; Tate et al., 1973). Reduced levels of GSH have been linked to increased sensitivity to ROS damage, resulting in mitochondrial swelling and subsequent damage (Chiou et al., 2010; Anderson, 1998). It has been found that agents that inhibit the glutathione/γ-glutamyl pathway in the bacteria from the Borrelia genus can be used to inhibit the growth and/or survival of bacteria from the Borrelia genus. In some embodiments, the presently disclosed subject matter provides a method for inhibiting the growth and/or survival of bacteria from the Borrelia genus, the method comprising contacting bacteria from the Borrelia genus with an effective amount of at least one agent that inhibits the glutathione/γ-glutamyl pathway in the bacteria from the Borrelia genus.

As used herein, the term “glutathione/γ-glutamyl pathway” refers to the pathway that involves glutathione synthesis, degradation, and use. For example, the glutathione/γ-glutamyl pathway encompasses the pathway by which glutathione is synthesized (using γ-glutamyl cysteine synthetase and glutathione synthetase), converted to glutamate (using γ-glutamyl transpeptidase, γ-glutamyl cyclotransferase, 5-oxoprolinase), reduced (using glutathione reductase) to form GSH, oxidized to form glutathione disulfide (GSSG), and transferred to the mitochondria by glutathione-s-transferase (GST), where it protects the mitochondria from ROS damage and functions in amino acid transport, such as by conjugation of GSH to other substrates, such as xenobiotic substrates.

It has been found that besides agents that enhance reactive oxygen species production (e.g., verteporfin, oltipraz, pyroglutamic acid, pidolic acid), agents that affect cell membranes (e.g., benzododecinium chloride, thonzonium bromide, zanamivir) and energy production (e.g., thonzonium bromide, oxantel) are more active against the B. burgdorferi persisters than the commonly used Lyme antibiotics that inhibit macromolecule biosynthesis.

The presently disclosed subject matter contemplates the use of various agents in connection with the methods, uses, and compositions described herein. Certain of the methods and compositions described herein relate to the inhibition of the glutathione/γ-glutamyl pathway using at least one agent described herein to inhibit the growth and/or survival of bacteria from the Borrelia genus and/or to treat Lyme disease. Aspects of the methods and compositions described herein relate to the use of least one agent described herein to inhibit the growth and/or survival of bacteria from the Borrelia genus and/or to treat Lyme disease.

As used herein, the phrase “inhibits the glutathione/γ-glutamyl pathway” refers to inhibition of activity of at least one component of the glutathione/γ-glutamyl pathway. It is contemplated herein that inhibition of the glutathione/γ-glutamyl pathway can occur via, for example, a receptor ligand (e.g., a small molecule, an antibody, a siRNA, a peptide), a ligand sequestrant (e.g., an antibody, a binding protein), a modulator of a pathway component or a combination of such modulators.

In some embodiments, the agent is selected from the group comprising small molecules, such as small organic or inorganic molecules; saccharides; oligosaccharides; polysaccharides; a biological macromolecule, such as peptides, proteins, peptide analogs and derivatives; peptidomimetics; nucleic acids, such as RNA interference molecules (e.g., siRNAs, shRNAs, antisense RNAs, ribozymes, dendrimers and aptamers); antibodies, including antibody fragments and intrabodies; an extract made from biological materials, such as bacteria, plants, fungi, animal cells, and animal tissues; naturally occurring or synthetic compositions; and any combination thereof. In some embodiments, at least one agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof. In some embodiments, at least one agent that inhibits the glutathione/γ-glutamyl pathway is selected from the group consisting of verteporfin, oltipraz, pyroglutamic acid, and dextrorphan tartrate.

As used herein, the term “small molecule” can refer to agents that are “natural product-like,” however, the term “small molecule” is not limited to “natural product-like” agents. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

As used herein, an “RNA interference molecule” refers to an agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

One of skill in the art can easily test an agent to determine if it inhibits the glutathione/γ-glutamyl pathway by assessing, for example, the levels of glutathione (GSH) present or synthesis of upstream or downstream proteins or enzymes controlled by the pathway in cultured cells and comparing the results to cells not treated with an agent. An agent is determined to be an inhibitor of the glutathione/γ-glutamyl pathway if the level of GSH or expression of or synthesis of upstream or downstream proteins or enzymes in a culture of cells is reduced by at least 20% compared to the level of GSH or expression or synthesis of upstream or downstream proteins or enzymes in cells that are cultured in the absence of the agent; preferably the level of GSH or expression or synthesis of upstream or downstream proteins or enzymes is altered by at least 30%, at least 40%, at least 50%, at least 60%, at least 70%, at least 80%, at least 90%, at least 95%, or at least 99% in the presence of an agent.

The terms “decrease”, “reduced”, “reduction”, “decrease” or “inhibit” are all used herein generally to mean a decrease by a statistically significant amount. However, for avoidance of doubt, “reduced”, “reduction”, “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In one embodiment, the decrease includes a 100% decrease (e.g. absent level as compared to a reference sample), or any decrease between 10-100% as compared to a reference level.

The terms “increased”, ‘increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

Certain methods, compositions, and agents contemplated herein inhibit the glutathione/γ-glutamyl pathway and/or the level of GSH. The methods, compositions, and agents contemplated herein can decrease glutathione/γ-glutamyl pathway activity and/or the level of GSH by at least about 10%, 20%, 30%, 40%, 50%, 60%, 70%, 80, 90%, or as much as 100%, at least about a 2-fold, or at least about a 3-fold, or at least about a 4-fold, or at least about a 5-fold or at least about a 10-fold decrease, or any decrease between 2-fold and 10-fold or greater as compared to a reference level (e.g., an objective measure of the level of GSH before employing the method, composition, and/or agent).

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

In some embodiments, at least one agent is a cell membrane disruptor, an energy inhibitor, and/or a reactive oxygen species (ROS) producer. As used herein, the term “cell membrane disruptor” refers to an agent, such as a peptide or small molecule, which interferes with the normal functioning of the cell membrane of a cell. Non-limiting examples of cell membrane disruptors include verteporfin, benzododecinium chloride, thonzonium bromide, and zanamivir. As used herein, the term “energy inhibitor” refers to an agent that inhibits energy production in a cell. A non-limiting example includes oxantel, a fumarate reductase inhibitor. As used herein, the term “reactive oxygen species (ROS)” refers to chemically reactive molecules that contain oxygen, such as oxygen ions and peroxides. ROS can cause significant damage to cell structures. As used herein, the terms “reactive oxygen species producer” and “ROS producer” refer to an agent that causes an increase in ROS production. Non-limiting examples of ROS producers include verteporfin, oltipraz, and pyroglutamic acid. In some embodiments, at least one agent is selected from the group consisting of verteporfin, oltipraz, pyroglutamic acid, zanamiver, oxantel, benzododecinium chloride, and thonzonium bromide.

In some embodiments, the presently disclosed subject matter provides a method for inhibiting the growth and/or survival of bacteria from the Borrelia genus, the method comprising contacting bacteria from the Borrelia genus with an effective amount of at least one agent selected from the group consisting of verteporfin, thonzonium bromide, tetrachloroethylene, benzododecinium chloride, butyl chloride (1-chlorobutane), 3-formyl rifamycin, potassium antimonyl tartrate (tartar emetic), toltrazuril, thiostrepton, pyroglutamic acid, mepartricin, tilorone, oxantel, hycanthone, pyrimethamine, trilocarban (3,4,4′-trichlorocarbanilide), carbenicillin, oltipraz, bitoscanate, sarafloxacin, bacitracin, dextrorphan tartrate, tetramisole, bifonazole, ethacridine lactate, zanamivir, aluminum lactate, p-arsanilic acid, nifursol, nevirapine, rifaximin, oxibendazole, metrifonate, indatraline, florfenicol, benznidazole, ganciclovir, tazobactam, oxfendazole, phenothiazine, flubendazole, midecamycin, fluconazole, docosanol, aztreonam, benzoylpas (4-Benzamido salicylic acid), trifluridine, undecylenic acid, closantel, cefixime, thiamphenicol, ricobendazole (albendazole oxide), sulfamoxole, clopidol, tosufloxacin, metampicillin, amikacin, lamivudine, cephalosporin C, sulfachlorpyridazine, lomofungin, artesunate, valacyclovir, carzenide (4-carboxybenzenesulfonamide), clinafloxacin, efavirenz, cefsulodin, cloxyquin (5-chloro-8-hydroxy-quinoline), symclosene (trichloroisocyanuric acid), didanosine (2′-3′-dideoxyinosine), floxuridine (5-fluorodeoxyuridine), cyacetacide, roxithromycin, oxiconazole nitrate, climbazole, protionamide, ribavirin, griseofulvin, rifamycin SV, salicylanilide, diclazuril, imiquimod, penciclovir, nystatin, ampicillin, puromycin, stavudine (2′,3′-didehydro-3′-deoxythymidine), potassium iodide, voriconazole, penimepicycline, amantadine, nitroxoline (8-hydroxy 5-nitroquinoline), 4-aminosalicylic acid, ciclopirox olamine, nelfinavir mesylate, anisomycin, betamipron (n-benzoyl-b-alanine), famciclovir, flucytosine (5-fluorocytosine), clotrimazole, rimantadine, pazufloxacin, carbadox, amantadine, dibekacin, clorsulon, thiacetazone (amithiozone), fleroxacin, clofoctol, butoconazole nitrate, quinaldine blue, and methylene blue.

In some embodiments, at least one agent is selected from the group consisting of verteporfin, 3-formyl rifamycin, rifaximin, rifamycin SV, sarafloxacin, clinafloxacin, tosufloxacin, fluconazole, climbazole, tilorone, artemisinin, potassium antimonyl tartrate tryhydrate, tartar emetic, closantel, toltrazuril, thiostrepton, mepartricin, tilorone, oxantel, pyroglutamic acid, hycanthone, pyrimethamine, carbenicillin, oltipraz, bitoscanate, sarafloxacin, bacitracin, dextrorphan tartrate, tetramisole, bifonazole, ethacridine lactate, zanamivir, oxibendazole, indatraline, nevirapine, ganciclovir, phenothiazine, oxfendazole, flubendazole, tazobactam, aztreonam, benzoylpas, fluconazole, cefixime, sulfamoxole, tosufloxacin, lamivudine, cefsulodin, didanosine, floxuridine, cyacetacide, oxiconazole, roxithromycin, ribavirin, griseofulvin, rifamycin SV, penciclovir, nystatin, penimepicycline, puromycin, quinaldine blue, and methylene blue.

In some embodiments, at least one agent is selected from the group consisting of verteporfin, oltipraz, pyroglutamic acid, dextrorphan tartrate, zanamivir, 3-formyl rifamycin, quinaldine blue, and methylene blue.

In some embodiments, the bacteria is contacted with at least one presently disclosed agent and at least one other agent that is known to be useful in treating Lyme disease, symptoms of Lyme disease, and/or effects or complications of Lyme disease. Examples of current agents used for Lyme disease include doxycycline, amoxicillin, cefuroxime, and ceftriaxone. In some embodiments, the bacteria is contacted with at least one presently disclosed agent and at least one other agent selected from the group consisting of doxycycline, amoxicillin, cefuroxime, ceftriaxone, metronidazole, tinidazole, erythromycin, azithromycin, clarithromycin, penicillin G, cefotaxime, a nonsteroidal anti-inflammatory agent, a corticosteroid, and a disease-modifying antirheumatic drug (DMARD). Non-limiting examples of nonsteroidal anti-inflammatory agents include ibuprofen and naproxen sodium. Non-limiting examples of disease-modifying antirheumatic drugs (DMARDs) include azathioprine; biologics, such as actemra, cimzia, enbrel, humira, kineret, orencia, remicade, rituxan, simponi; cyclophosphamide, cyclosporine, hydroxychloroquine, leflunomide, methotrexate, sulfasalazine, and tofacitinib.

In some embodiments, at least one of the presently disclosed agents can be used in combination with at least one previously identified agent from the 27 agents identified in our previous study (Feng J, Wang T, Shi W, Zhang S, Sullivan D, Auwaerter P G, 2014). In some embodiments, at least one of the presently disclosed agents can be used in combination with at least one previously identified agent from the 27 agents identified in our previous study (Feng J, Wang T, Shi W, Zhang S, Sullivan D, Auwaerter P G, 2014) and at least one other agent that is known to be useful in treating Lyme disease, symptoms of Lyme disease, and/or effects or complications of Lyme disease. The 27 agents include daptomycin, clofazimine, cefoperazone, carbomycin, vancomycin, cephalothin, cefotiam, cefmetazole, cefepime, amodiaquin, streptomycin, ticarcillin, cefonicid, piperacillin-tazobactam, cefdinir, ceforanide, cefmenoxime, bismuth, ceftizoxime, ceftibuten, amphotericin B, cefamandole, quinine hydrobromide, cyclacillin, collistin, sulfameter, and tigecycline. In some aspects, one or more of the 27 agents are excluded from use in the presently disclosed compositions, kits and methods, and/or is excluded from use in combination with at least one of the presently disclosed agents. In some embodiments, at least one agent is not daptomycin. In some embodiments, daptomycin is not used in combination with at least one of the presently disclosed agents. In some embodiments, at least one agent is not clofazimine. In some embodiments, clofazimine is not used in combination with at least one of the presently disclosed agents. In some embodiments, at least one agent is not cefoperazone. In some embodiments, cefoperazone is not used in combination with at least one of the presently disclosed agents. In some embodiments, at least one agent is not carbomycin. In some embodiments, carbomycin is not used in combination with at least one of the presently disclosed agents. In some embodiments, at least one agent is not vancomycin. In some embodiments, vancomycin is not used in combination with at least one of the presently disclosed agents. In some embodiments, at least one agent is not cephalothin. In some embodiments, cephalothin is not used in combination with at least one of the presently disclosed agents. In some embodiments, at least one agent is not cefotiam. In some embodiments, cefotiam is not used in combination with at least one of the presently disclosed agents. In some embodiments, at least one agent is not cefmetazole. In some embodiments, cefmetazole is not used in combination with at least one of the presently disclosed agents. In some embodiments, at least one agent is not cefepime. In some embodiments, cefepime is not used in combination with at least one of the presently disclosed agents. In some embodiments, at least one agent is not amodiaquin. In some embodiments, amodiaquin is not used in combination with at least one of the presently disclosed agents. In some embodiments, at least one agent is not streptomycin. In some embodiments, streptomycin is not used in combination with at least one of the presently disclosed agents. In some embodiments, at least one agent is not ticarcillin. In some embodiments, ticarcillin is not used in combination with at least one of the presently disclosed agents. In some embodiments, at least one agent is not cefonicid. In some embodiments, cefonicid is not used in combination with at least one of the presently disclosed agents. In some embodiments, at least one agent is not piperacillin-tazobactam. In some embodiments, piperacillin-tazobactam is not used in combination with at least one of the presently disclosed agents. In some embodiments, at least one agent is not cefdinir. In some embodiments, cefdinir is not used in combination with at least one of the presently disclosed agents. In some embodiments, at least one agent is not ceforanide. In some embodiments, ceforanide is not used in combination with at least one of the presently disclosed agents. In some embodiments, at least one agent is not cefmenoxime. In some embodiments, cefmenoxime is not used in combination with at least one of the presently disclosed agents. In some embodiments, at least one agent is not bismuth. In some embodiments, bismuth is not used in combination with at least one of the presently disclosed agents. In some embodiments, at least one agent is not ceftizoxime. In some embodiments, ceftizoxime is not used in combination with at least one of the presently disclosed agents. In some embodiments, at least one agent is not ceftibuten. In some embodiments, ceftibuten is not used in combination with at least one of the presently disclosed agents. In some embodiments, at least one agent is not amphotericin B. In some embodiments, amphotericin B is not used in combination with at least one of the presently disclosed agents.

In some embodiments, at least one agent is not cefamandole. In some embodiments, cefamandole is not used in combination with at least one of the presently disclosed agents. In some embodiments, at least one agent is not quinine hydrobromide. In some embodiments, quinine hydrobromide is not used in combination with at least one of the presently disclosed agents. In some embodiments, at least one agent is not cyclacillin. In some embodiments, cyclacillin is not used in combination with at least one of the presently disclosed agents. In some embodiments, at least one agent is not collistin. In some embodiments, collistin is not used in combination with at least one of the presently disclosed agents. In some embodiments, at least one agent is not sulfameter. In some embodiments, sulfameter is not used in combination with at least one of the presently disclosed agents. In some embodiments, at least one agent is not tigecycline. In some embodiments, tigecycline is not used in combination with at least one of the presently disclosed agents.

Borrelia is a genus of bacteria of the Spirochete phylum. The Borrelia burgdorferi sensu lato complex includes at least 18 genospecies. Non-limiting examples of bacteria in this genus include B. burgdorferi, B. garinii, B. afzelii, B. americana, B. carolinensis, B. lusitaniae, B. japonica, B. miyamotoii and B. sinica. In some embodiments, the bacteria are Borrelia burgdorferi. In some embodiments, the Borrelia burgdorferi comprise a morphological form selected from the group consisting of a spirochete form, a spheroplast form, a cystic or round body form, a microcolony form, a biofilm-like and biofilm form, and combinations thereof. In some embodiments, the bacteria comprise a morphological form of Borrelia burgdorferi selected from the group consisting of round bodies, planktonic, and biofilm.

In some embodiments, the bacteria comprise replicating forms of Borrelia burgdorferi, non-replicating persister forms of Borrelia burgdorferi, and combinations of replicating forms of Borrelia burgdorferi and non-replicating persister forms of Borrelia burgdorferi. As used herein, the term “non-replicating persister cells,” refers to bacterial cells that enter a state in which they stop replicating and are able to tolerate antibiotics.

The term “contacting” as used herein refers to any action that results in at least one compound of the presently disclosed subject matter physically contacting at least one bacterial cell or the environment in which at least one bacterial cell resides (e.g., a culture medium). In some embodiments, contacting occurs in vitro or in vivo.

In some embodiments, contacting bacteria from the Borrelia genus with an effective amount of at least one agent inhibits the growth and/or survival of the population of non-replicating persister forms of bacteria by at least about 10%, 20%, 30%, 40%, 50%, 60%, 70%, 80, 90%, or as much as 100%. In some embodiments, at least one agent inhibits the growth and/or survival of greater than about 35 percent of the population of non-replicating persister forms of bacteria. In some embodiments, at least one agent inhibits the growth and/or survival of greater than about 50 percent of the population of non-replicating persister forms of bacteria.

### II Methods for Treating Lyme Disease

In some embodiments, the presently disclosed subject matter provides a method for treating Lyme disease in a patient in need thereof, the method comprising administering to a patient an effective amount of at least one agent that inhibits the glutathione/γ-glutamyl pathway in the patient. In some embodiments, at least one agent that inhibits the glutathione/γ-glutamyl pathway is selected from the group consisting of verteporfin, oltipraz, pyroglutamic acid, and dextrorphan tartrate. In some embodiments, the method of treating Lyme disease comprises administering at least one agent that inhibits the glutathione/γ-glutamyl pathway with the proviso that the at least one agent is not verteporfin. In some embodiments, the method of treating Lyme disease comprises administering at least one agent that inhibits the glutathione/γ-glutamyl pathway with the proviso that the at least one agent is not oltipraz. In some embodiments, the method of treating Lyme disease comprises administering at least one agent that inhibits the glutathione/γ-glutamyl pathway with the proviso that the at least one agent is not pyroglutamic acid. In some embodiments, the method of treating Lyme disease comprises administering at least one agent that inhibits the glutathione/γ-glutamyl pathway with the proviso that the at least one agent is not dextrorphan tartrate.

In some embodiments, at least one agent is a cell membrane disruptor, an energy inhibitor, and/or a reactive oxygen species (ROS) producer. In some embodiments, at least one agent is selected from the group consisting of verteporfin, oltipraz, pyroglutamic acid, zanamiver, oxantel, benzododecinium chloride, and thonzonium bromide.

In some embodiments, the presently disclosed subject matter provides a method for treating Lyme disease in a patient in need thereof, the method comprising administering to a patient an effective amount of at least one agent selected from the group consisting of verteporfin, thonzonium bromide, tetrachloroethylene, benzododecinium chloride, butyl chloride (1-chlorobutane), 3-formyl rifamycin, potassium antimonyl tartrate (tartar emetic), toltrazuril, thiostrepton, pyroglutamic acid, mepartricin, tilorone, oxantel, hycanthone, pyrimethamine, trilocarban (3,4,4′-trichlorocarbanilide), carbenicillin, oltipraz, bitoscanate, sarafloxacin, bacitracin, dextrorphan tartrate, tetramisole, bifonazole, ethacridine lactate, zanamivir, aluminum lactate, p-arsanilic acid, nifursol, nevirapine, rifaximin, oxibendazole, metrifonate, indatraline, florfenicol, benznidazole, ganciclovir, tazobactam, oxfendazole, phenothiazine, flubendazole, midecamycin, fluconazole, docosanol, aztreonam, benzoylpas (4-Benzamido salicylic acid), trifluridine, undecylenic acid, closantel, cefixime, thiamphenicol, ricobendazole (albendazole oxide), sulfamoxole, clopidol, tosufloxacin, metampicillin, amikacin, lamivudine, cephalosporin C, sulfachlorpyridazine, lomofungin, artesunate, valacyclovir, carzenide (4-carboxybenzenesulfonamide), clinafloxacin, efavirenz, cefsulodin, cloxyquin (5-chloro-8-hydroxy-quinoline), symclosene (trichloroisocyanuric acid), didanosine (2′-3′-dideoxyinosine), floxuridine (5-fluorodeoxyuridine), cyacetacide, roxithromycin, oxiconazole nitrate, climbazole, protionamide, ribavirin, griseofulvin, rifamycin SV, salicylanilide, diclazuril, imiquimod, penciclovir, nystatin, ampicillin, puromycin, stavudine (2′,3′-didehydro-3′-deoxythymidine), potassium iodide, voriconazole, penimepicycline, amantadine, nitroxoline (8-hydroxy 5-nitroquinoline), 4-aminosalicylic acid, ciclopirox olamine, nelfinavir mesylate, anisomycin, betamipron (n-benzoyl-b-alanine), famciclovir, flucytosine (5-fluorocytosine), clotrimazole, rimantadine, pazufloxacin, carbadox, amantadine, dibekacin, clorsulon, thiacetazone (amithiozone), fleroxacin, clofoctol, butoconazole nitrate, quinaldine blue, and methylene blue. In some embodiments, at least one agent is selected from the group consisting of verteporfin, 3-formyl rifamycin, rifaximin, rifamycin SV, sarafloxacin, clinafloxacin, tosufloxacin, fluconazole, climbazole, tilorone, artemisinin, potassium antimonyl tartrate tryhydrate, tartar emetic, closantel, toltrazuril, thiostrepton, mepartricin, tilorone, oxantel, pyroglutamic acid, hycanthone, pyrimethamine, carbenicillin, oltipraz, bitoscanate, sarafloxacin, bacitracin, dextrorphan tartrate, tetramisole, bifonazole, ethacridine lactate, zanamivir, oxibendazole, indatraline, nevirapine, ganciclovir, phenothiazine, oxfendazole, flubendazole, tazobactam, aztreonam, benzoylpas, fluconazole, cefixime, sulfamoxole, tosufloxacin, lamivudine, cefsulodin, didanosine, floxuridine, cyacetacide, oxiconazole, roxithromycin, ribavirin, griseofulvin, rifamycin SV, penciclovir, nystatin, penimepicycline, puromycin, quinaldine blue, and methylene blue.

In some aspects, the presently disclosed methods, compositions, and kits exclude one or more agents. In some embodiments, at least one agent is not verteporfin. In some embodiments, at least one agent is not oltipraz. In some embodiments, at least one agent is not pyroglutamic acid. In some embodiments, at least one agent is not, dextrorphan tartrate. In some embodiments, at least one agent is not zanamivir. In some embodiments, at least one agent is not butyl chloride. In some embodiments, at least one agent is not. In some embodiments, at least one agent is not 3-formyl rifamycin. In some embodiments, at least one agent is not quinaldine blue. In some embodiments, at least one agent is not methylene blue. In some embodiments, at least one agent is not verteporfin. In some embodiments, at least one agent is not thonzonium bromide. In some embodiments, at least one agent is not tetrachloroethylene. In some embodiments, at least one agent is not benzododecinium chloride. In some embodiments, at least one agent is not butyl chloride (1-chlorobutane). In some embodiments, at least one agent is not potassium antimonyl tartrate (tartar emetic). In some embodiments, at least one agent is not toltrazuril. In some embodiments, at least one agent is not thiostrepton. In some embodiments, at least one agent is not pyroglutamic acid. In some embodiments, at least one agent is not mepartricin. In some embodiments, at least one agent is not tilorone. In some embodiments, at least one agent is not oxantel. In some embodiments, at least one agent is not hycanthone. In some embodiments, at least one agent is not pyrimethamine. In some embodiments, at least one agent is not trilocarban (3,4,4′-trichlorocarbanilide). In some embodiments, at least one agent is not carbenicillin. In some embodiments, at least one agent is not oltipraz. In some embodiments, at least one agent is not bitoscanate. In some embodiments, at least one agent is not sarafloxacin. In some embodiments, at least one agent is not bacitracin. In some embodiments, at least one agent is not dextrorphan tartrate. In some embodiments, at least one agent is not tetramisole. In some embodiments, at least one agent is not bifonazole. In some embodiments, at least one agent is not ethacridine lactate. In some embodiments, at least one agent is not zanamivir. In some embodiments, at least one agent is not aluminum lactate. In some embodiments, at least one agent is not p-arsanilic acid. In some embodiments, at least one agent is not nifursol. In some embodiments, at least one agent is not nevirapine. In some embodiments, at least one agent is not rifaximin. In some embodiments, at least one agent is not oxibendazole. In some embodiments, at least one agent is not metrifonate. In some embodiments, at least one agent is not indatraline. In some embodiments, at least one agent is not florfenicol. In some embodiments, at least one agent is not benznidazole. In some embodiments, at least one agent is not ganciclovir. In some embodiments, at least one agent is not tazobactam. In some embodiments, at least one agent is not oxfendazole. In some embodiments, at least one agent is not phenothiazine. In some embodiments, at least one agent is not flubendazole. In some embodiments, at least one agent is not midecamycin. In some embodiments, at least one agent is not fluconazole. In some embodiments, at least one agent is not docosanol. In some embodiments, at least one agent is not aztreonam. In some embodiments, at least one agent is not benzoylpas (4-Benzamido salicylic acid). In some embodiments, at least one agent is not trifluridine. In some embodiments, at least one agent is not undecylenic acid. In some embodiments, at least one agent is not closantel. In some embodiments, at least one agent is not cefixime. In some embodiments, at least one agent is not thiamphenicol. In some embodiments, at least one agent is not ricobendazole (albendazole oxide). In some embodiments, at least one agent is not sulfamoxole. In some embodiments, at least one agent is not clopidol. In some embodiments, at least one agent is not tosufloxacin. In some embodiments, at least one agent is not metampicillin. In some embodiments, at least one agent is not amikacin. In some embodiments, at least one agent is not lamivudine. In some embodiments, at least one agent is not cephalosporin C. In some embodiments, at least one agent is not sulfachlorpyridazine. In some embodiments, at least one agent is not lomofungin. In some embodiments, at least one agent is not artesunate. In some embodiments, at least one agent is not valacyclovir. In some embodiments, at least one agent is not carzenide (4-carboxybenzenesulfonamide). In some embodiments, at least one agent is not clinafloxacin. In some embodiments, at least one agent is not efavirenz. In some embodiments, at least one agent is not cefsulodin. In some embodiments, at least one agent is not cloxyquin (5-chloro-8-hydroxy-quinoline). In some embodiments, at least one agent is not symclosene (trichloroisocyanuric acid). In some embodiments, at least one agent is not didanosine (2′-3′-dideoxyinosine). In some embodiments, at least one agent is not floxuridine (5-fluorodeoxyuridine). In some embodiments, at least one agent is not cyacetacide. In some embodiments, at least one agent is not roxithromycin. In some embodiments, at least one agent is not oxiconazole nitrate. In some embodiments, at least one agent is not climbazole. In some embodiments, at least one agent is not protionamide. In some embodiments, at least one agent is not ribavirin. In some embodiments, at least one agent is not griseofulvin. In some embodiments, at least one agent is not rifamycin SV. In some embodiments, at least one agent is not salicylanilide. In some embodiments, at least one agent is not diclazuril. In some embodiments, at least one agent is not imiquimod. In some embodiments, at least one agent is not penciclovir. In some embodiments, at least one agent is not nystatin. In some embodiments, at least one agent is not ampicillin. In some embodiments, at least one agent is not puromycin. In some embodiments, at least one agent is not stavudine (2′,3′-didehydro-3′-deoxythymidine). In some embodiments, at least one agent is not potassium iodide. In some embodiments, at least one agent is not voriconazole. In some embodiments, at least one agent is not penimepicycline. In some embodiments, at least one agent is not amantadine. In some embodiments, at least one agent is not nitroxoline (8-hydroxy 5-nitroquinoline). In some embodiments, at least one agent is not 4-aminosalicylic acid. In some embodiments, at least one agent is not ciclopirox olamine. In some embodiments, at least one agent is not nelfinavir mesylate. In some embodiments, at least one agent is not anisomycin. In some embodiments, at least one agent is not betamipron (n-benzoyl-b-alanine). In some embodiments, at least one agent is not famciclovir. In some embodiments, at least one agent is not flucytosine (5-fluorocytosine). In some embodiments, at least one agent is not clotrimazole. In some embodiments, at least one agent is not rimantadine. In some embodiments, at least one agent is not pazufloxacin. In some embodiments, at least one agent is not carbadox. In some embodiments, at least one agent is not amantadine. In some embodiments, at least one agent is not dibekacin. In some embodiments, at least one agent is not clorsulon. In some embodiments, at least one agent is not thiacetazone (amithiozone). In some embodiments, at least one agent is not fleroxacin. In some embodiments, at least one agent is not clofoctol. In some embodiments, at least one agent is not butoconazole nitrate. In some embodiments, at least one agent is not quinaldine blue. In some embodiments, at least one agent is not methylene blue.

In some embodiments, at least one agent is selected from the group consisting of verteporfin, 3-formyl rifamycin, rifaximin, rifamycin SV, sarafloxacin, clinafloxacin, tosufloxacin, fluconazole, climbazole, tilorone, artemisinin, potassium antimonyl tartrate tryhydrate, tartar emetic, closantel, toltrazuril, thiostrepton, mepartricin, tilorone, oxantel, pyroglutamic acid, hycanthone, pyrimethamine, carbenicillin, oltipraz, bitoscanate, sarafloxacin, bacitracin, dextrorphan tartrate, tetramisole, bifonazole, ethacridine lactate, zanamivir, oxibendazole, indatraline, nevirapine, ganciclovir, phenothiazine, oxfendazole, flubendazole, tazobactam, aztreonam, benzoylpas, fluconazole, cefixime, sulfamoxole, tosufloxacin, lamivudine, cefsulodin, didanosine, floxuridine, cyacetacide, oxiconazole, roxithromycin, ribavirin, griseofulvin, rifamycin SV, penciclovir, nystatin, penimepicycline, puromycin, quinaldine blue, and methylene blue.

In some embodiments, at least one presently disclosed agent is combined with at least one other agent that is known to be useful in treating Lyme disease, symptoms of Lyme disease, and/or effects or complications of Lyme disease. In some embodiments, the patient is administered at least one presently disclosed agent and at least one other agent selected from the group consisting of doxycycline, amoxicillin, cefuroxime, ceftriaxone, metronidazole, tinidazole, erythromycin, azithromycin, clarithromycin, penicillin G, cefotaxime, a nonsteroidal anti-inflammatory agent, a corticosteroid, and a disease-modifying antirheumatic drug (DMARD).

In some embodiments, the bacteria are Borrelia burgdorferi. In some embodiments, the bacteria comprise replicating forms of Borrelia burgdorferi, non-replicating persister forms of Borrelia burgdorferi, and combinations of replicating forms of Borrelia burgdorferi and non-replicating persister forms of Borrelia burgdorferi. In some embodiments, the bacteria comprise a morphological form of Borrelia burgdorferi selected from the group consisting of round bodies, planktonic, and biofilm.

In some embodiments, the patient has, or is suspected of having, post-treatment Lyme disease syndrome (PTLDS) and/or antibiotic refractory Lyme arthritis. In some embodiments, the patient is a human.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

In some embodiments, the term “effective amount” refers to the amount of agent required to inhibit or kill a bacterial cell. In other embodiments, the term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired at least biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy. In particular embodiments, the disease, disorder, or condition is Lyme disease.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

The term “combination” is used in its broadest sense and means that a subject is administered at least two agents. More particularly, the term “in combination” refers to the concomitant administration of two (or more) active agents for the treatment of a, e.g., single and multiple disease states with heterogeneous bacterial populations consisting of growing and non-growing or any in between bacterial cells. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one, but not the other). The single dosage form may include additional active agents for the treatment of the disease state.

Further, the compounds described herein can be administered alone or in combination with adjuvants that enhance stability of the compounds, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the agents can be varied so long as the beneficial effects of the combination of these agents are achieved. Accordingly, the phrase “in combination with” refers to the administration of at least two agents either simultaneously, sequentially, or a combination thereof. Therefore, a subject administered a combination of at least two agents can receive one agent and at least one additional agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the agents are administered simultaneously, they can be administered to the subject as separate pharmaceutical compositions or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of at least two agents is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In therapeutic and/or diagnostic applications, the agents of the disclosure (e.g., agents that inhibit the glutathione/γ-glutamyl pathway) can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the agents of the present disclosure, in particular, those formulated as solutions, may be administered parenterally, such as by intravenous injection. The agents can be formulated readily using pharmaceutically acceptable carriers well known in the art into dosages suitable for oral administration. Such carriers enable the agents of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these agents may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### 1. INTRODUCTION

Borrelia burgdorferi is the causative agent of Lyme disease, the most common vector borne disease in the United States and Europe. Although about 27,000 confirmed cases of Lyme disease in the United States were reported to the CDC in 2013, the total number of cases is estimated to be as high as 300,000 each year (CDC, “Lyme Disease”, 2015; Hinckley et al., 2014). B. burgdorferi is transmitted during blood feeding of Ixodes ticks on hosts including rodents, small mammals and humans (Radolf et al., 2012). Lyme disease in humans is a multi-system disorder whose early stage is characterized by erythema migrans, a rapidly spreading rash that appears at the cutaneous site of infection in about 50% of patients (Wormser et al., 2006). Upon bacterial dissemination, patients can experience severe symptoms such as arthritis, carditis and neurologic impairment (Wormser et al., 2006).

The current treatment for Lyme disease is a 2-4 week antibiotic monotherapy with doxycycline, amoxicillin or cefuroxime axetil (Wormser et al., 2006). However, at least 20% of patients receiving this treatment experience chronic symptoms such as fatigue, muscle pain, and neurological impairment even six months after treatment (CDC, “Post Treatment Lyme Disease”, 2015; Feder et al., 2007). Patients with these symptoms are diagnosed with Post Treatment Lyme Disease Syndrome (PTLDS) and report significantly impaired functional ability and lower quality of life compared to Lyme patients without these symptoms (Aucott et al., 2013). The cause of PTLDS is unknown. Several theories have been proposed to explain this syndrome, including host response to continued presence of bacterial debris, autoimmunity, co-infections, and bacterial persisters not killed by the current Lyme antibiotics (Phillips et al., 1998).

Evidence that supports the continued presence of persisting organisms despite antibiotic treatment has been well documented in various animal models such as mice, dogs and nonhuman primates (Barthold et al., 2010; Embers et al., 2012; Hodzic et al., 2014; Straubinger et al., 1997). Intriguingly, the organism could not be cultured in conventional culture medium after antibiotic treatment but could be detected by more sensitive and indirect techniques such as xenodiagnosis and PCR. Similarly, in patients with chronic Lyme infections, signs of persisting organisms in a nonculturable form could be detected by positive PCR and xenodiagnosis (Marques et al., 2014). Persistent bacteria are suggested as an explanation for the chronic symptoms of PTLDS as well as the observations of B. burgdorferi DNA without positive culturing results (Hodzic et al., 2008; Bayer et al., 1996).

Persisters are a heterogeneous bacterial subpopulation that are genetically drug susceptible but have phenotypic variations that for surviving in the presence of stressors such as antibiotics (Zhang et al., 2014). B. burgdorferi can change morphologies as the culture ages (Feng J, Wang T, Shi W, Zhang S, Sullivan D, Auwaerter P G, 2014; Feng J, Auwaerter P G, Zhang Y, 2015). Log phase culture of B. burgdorferi consists primarily of spirochetes but round bodies and microcolonies become more abundant as the culture reaches stationary phase (Feng J, Wang T, Shi W, Zhang S, Sullivan D, Auwaerter P G, 2014; Feng J, Auwaerter P G, Zhang Y, 2015). The current Lyme antibiotics while having high activity against the spirochete log phase bacteria, show little activity against the stationary phase morphological variants (Feng J, Wang T, Shi W, Zhang S, Sullivan D, Auwaerter P G, 2014; Feng J, Auwaerter P G, Zhang Y, 2015; Sapi et al., 2011).

To identify drugs that target B. burgdorferi persisters, we recently screened an FDA drug library and identified 165 hits with higher activity against B. burgdorferi persisters than the currently used Lyme antibiotics amoxicillin and doxycycline (Feng J, Wang T, Shi W, Zhang S, Sullivan D, Auwaerter P G, 2014). In that study, which is disclosed in PCT Patent Application No. PCT/US2015/024122 and is hereby incorporated by reference in its entirety, we reported the top 27 hits and some top hits were further evaluated in drug combination studies (Feng J, Wang T, Shi W, Zhang S, Sullivan D, Auwaerter P G, 2014; Feng J, Auwaerter P G, Zhang Y, 2015). In this study, we present findings on the remainder of the 113 drug candidates from the FDA drug library with higher activity against the B. burgdorferi stationary phase culture than amoxicillin and doxycycline.

### 2. RESULTS AND DISCUSSION

2.1. Identification of Drug Candidates with High Anti-Persister Activity

We identified 165 hits with higher activity against B. burgdorferi persisters than the currently used Lyme antibiotics. We further analyzed and characterized these active hits. By removing redundant hits from the library and also those that could not be repeated, we obtained 113 that gave consistent results (Table 3). Of the 113 hits, the top 50 candidates that can be used in humans and killed 65% or more of the stationary phase bacteria according to either the SYBR/PI assay or microscopic quantitation are presented in Table 1. The remainder of active hits that may not be used in humans and also less active ones are presented in Table 3. However, these agents in general were not as active as the top hits such as daptomycin, clofazimine, cefoperazone, and anthracyclines from the previous screens (Feng J, Wang T, Shi W, Zhang S, Sullivan D, Auwaerter P G, 2014; Feng J, Shi W, Zhang S, Zhang Y, 2015). These active hits are grouped into antimicrobial agents (antibiotics, antivirals, antifungals, anthelmintics or antiparasitics), agents used for treating other disease conditions, as well as agents that may only be used as topical agents or not used internally and are presented below (Table 1).

In our previous study, we compared the activity of antibiotics against non-growing persisters with their activity against growing B. burgdorferi. Here we also tested the MICs of some active hits. We found these drugs showed good activity against the growing B. burgdorferi with a low MIC (Table 2).

TABLE 1
Top 50 active hits with better activity (p-value < 0.05 or live percentage by
microscopy assay less 68%) against B. burgdorferi stationary phase cells than
current Lyme antibiotics^{a}.

| | | | Residual | |
|---|---|---|---|---|
| | | Residual | viable cells | |
| | | viable cells | (SYBR | |
| Drugs (50 μM) | Category | (Microscopy)^{b} | Green/PI)^{c} | p-value^{d} |
| Control (no drug) | | 93% | 94% | — |
| Doxycycline | Lyme antibiotic | 75% | 67% | 0.23360 |
| Amoxicillin | Lyme antibiotic | 76% | 76% | 1.00000 |
| Cefuroxime | Lyme antibiotic | 49% | 43% | 0.00032 |
| Daptomycin | Antibiotic | 35% | 28% | 0.00001 |
| Verteporfin^{d} | Ophthalmic | 47% | 27% | 0.00284 |
| 3-formyl Rifamycin | Antibacterial | 59% | 42% | 0.00103 |
| Tartar emetic | Anthelmintic | 45% | 42% | 0.00250 |
| Toltrazuril | Antiprotozoal | 60% | 43% | 0.00296 |
| | (Coccidostat) | | | |
| Thiostrepton | Antibiotic | 66% | 43% | 0.00131 |
| Mepartricin | Antifungal | 60% | 43% | 0.03214 |
| Tilorone | Antiviral | | 44% | 0.04955 |
| Oxantel | Anthelmintic | 63% | 44% | 0.01599 |
| Pidolic acid^{e} | Antiseptic | 45% | 45% | 0.00477 |
| Hycanthone | Anthelmintic | | 45% | 0.00154 |
| Pyrimethamine | Antiprotozoal | 55% | 45% | 0.00030 |
| Carbenicillin | Antibiotic | 64% | 46% | 0.06453 |
| Oltipraz | Antitumor | 55% | 46% | 0.00121 |
| Bitoscanate | Anthelmintic | | 46% | 0.00730 |
| Sarafloxacin | Antibiotic | 50% | 47% | 0.05063 |
| Bacitracin | Antibiotic | 60% | 47% | 0.06536 |
| Dextrorphan | Analgesic | 43% | 47% | 0.00361 |
| tartrate^{e} | | | | |
| Tetramisole | Anthelmintic | | 48% | 0.07051 |
| Bifonazole | Antifungal | 50% | 48% | 0.09243 |
| Ethacridine lactate | Antiseptic | | 48% | 0.04619 |
| Zanamivir | Antiviral | 60% | 49% | 0.01224 |
| Artemesinin | Antimalarial | 45% | 49% | 0.10432 |
| Oxibendazole | Anthelminthic | | 51% | 0.00895 |
| Indatraline^{e} | Antidepressant | 43% | 51% | 0.02578 |
| Nevirapine | Antiviral | | 51% | 0.01604 |
| Ganciclovir | Antiviral | | 53% | 0.04466 |
| Phenothiazine | Anthelminthic | 53% | 54% | 0.00228 |
| Oxfendazole | Anthelminthic | | 54% | 0.00095 |
| Flubendazole | Anthelminthic | | 54% | 0.01183 |
| Tazobactam | Antibiotic | 56% | 54% | 0.10052 |
| Aztreonam | Antibiotic | 50% | 55% | 0.08105 |
| Benzoylpas | Antibiotic | | 55% | 0.04017 |
| Fluconazole | Antifungal | 45% | 55% | 0.10643 |
| Cefixime | Antibiotic | 56% | 56% | 0.03238 |
| Sulfamoxole | Antibiotic | 55% | 57% | 0.02541 |
| Tosufloxacin | Antibiotic | | 57% | 0.00067 |
| Lamivudine | Antiviral | | 58% | 0.01121 |
| Cefsulodin | Antibiotic | | 60% | 0.01463 |
| Didanosine | Antiviral | | 61% | 0.02494 |
| Floxuridine | Antiviral | | 61% | 0.01935 |
| Cyacetacide | Antibacterial | | 61% | 0.03407 |
| Oxiconazole nitrate | Antifungal | | 62% | 0.04957 |
| Roxithromycin | Antibiotic | 65% | 62% | 0.15382 |
| Ribavirin | Antiviral | | 63% | 0.00801 |
| Griseofulvin | Antifungal | | 63% | 0.00957 |
| Rifamycin sv | Antibiotic | 60% | 63% | 0.01872 |
| Penciclovir | Antiviral | 60% | 64% | 0.09707 |
| Nystatin | Antifungal | | 64% | 0.03312 |
| Penimepicycline | Antibiotic | 60% | 65% | 0.00972 |
| Puromycin | Antibiotic | 48% | 65% | 0.29297 |
| Quinaldine blue | Antimalarial | 35% | Over range^{f} | — |
| Methylene blue | Antimethemoglobinemic | 40% | Over range^{f} | — |
| hydrate | | | | |

^{a}Stationary phase B. burgdorferi (7 day old) cells were treated with drugs for 7 days. Daptomycin was used as a positive control with known high activity against B. burgdorferi persisters as shown previously. Drugs with live percentage of B. burgdorferi less than 65% by microscopy after drug exposure are presented in the table.
^{b}Residual viable B. burgdorferi was assayed by epifluorescence microscope counting.
^{c}Residual viable B. burgdorferi was calculated according to the regression equation and ratio of Green/Red fluorescence obtained by SYBR Green I/PI assay. Three images of each sample were captured and quantitatively analyzed to determine the mean percent residual cells as indicated.
^{d}p-values of the standard t-test for the treated groups (n = 3) versus a control group treated with amoxicillin, which is known to have poor activity against stationary-phase persisters.
^{e}The italicized drugs are used to treat other disease than infection.
^{f}The value is over that of the drug free control due to color of the compounds.

TABLE 2
MIC values of some active hits for B. burgdorferi.

| Active hits | MIC (μg/ml) |
|---|---|
| Verteporfin | 4.49-8.98 |
| Thonzonium Bromide | 0.92-1.85 |
| Benzododecinium Chloride | 0.60-1.20 |
| 3-formyl Rifamycin | 2.27-4.54 |
| Pidolic acid | 0.81-1.61 |
| Oltipraz | 0.71-1.41 |
| Fluconazole | 0.48-0.96 |
| Dextrorphan Tartrate | 1.27-2.55 |
| Quinaldine Blue | 2.43-4.86 |

2.2. Antimicrobial Agents with High Activity Against Stationary Phase B. burgdorferi

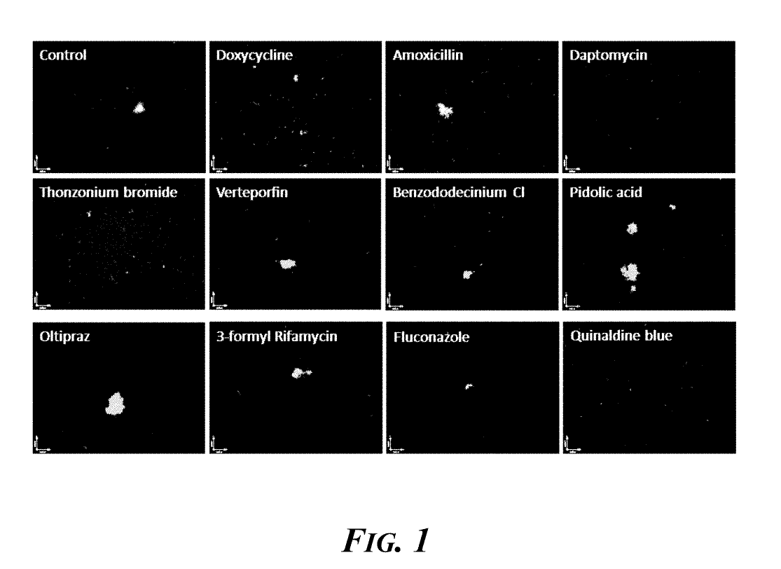

Readily available drugs with low toxicity are an important objective in this screen, as they are the most likely to be used for clinical treatment of Lyme disease. Here we focused on antimicrobial agents used in humans that had higher activity against the stationary phase B. burgdorferi than the commonly used Lyme antibiotics. The antibacterial agents include rifamycins (3-formal-rifamycin, rifaximin, rifamycin SV) (FIG. 1), thiostrepton, quinolone drugs (sarafloxacin, clinafloxacin, tosufloxacin), carbenicillin, tazobactam, aztreonam, puromycin (Table 1, Table 3). Some antifungal agents such as fluconazole (FIG. 1), mepartricin, bifonazole, climbazole, oxiconazole, nystatin, had reasonable activity against stationary phase B. burgdorferi (Table 1, Table 3). Antiviral agents zanamivir, nevirapine, tilorone (orally active interferon inducer) had good activity against stationary phase B. burgdorferi. Antimalarial agents artemisinin, methylene blue, and quidaldine blue were found to have good activity against stationary phase B. burgdorferi (Table 1). Antihelmintic and antiparasitic agents that had activity against B. burgdorferi included toltrazuril, tartar emetic, potassium antimonyl tartrate trihydrate, oxantel, closantel, hycanthone, pyrimethamine, and tetramisole (Table 1). These drugs with high activity against stationary phase B. burgdorferi in vitro are good potential candidates for drug combination studies and for further evaluation in animal models.

As previously published, the SYBR Green I/PI assay is a high-throughput technique that uses the ratio of green:red fluorescence in each sample to quantitate the amount of residual viable cells remaining (Feng J, Wang T, Shi W, Zhang S, Sullivan D, Auwaerter P G, 2014). While this technique has the benefits of high-throughput analysis, discoloration of the culture medium by test drugs can result in altered readings (Feng J, Wang T, Shi W, Zhang S, Sullivan D, Auwaerter P G, 2014). Quinaldine blue and methylene blue are two drugs whose staining properties resulted in medium discoloration and required verification through microscopy. Careful microscopy analysis revealed that quinaldine blue and methylene blue had high activity against B. burgdorferi persisters (Table 1, FIG. 1). Quinaldine (2-methylquinoline) is a heterocyclic quinoline compound that is used as an antimalarial and dye manufacturing, food colorants, pH indicators and pharmaceuticals. Methylene blue was originally used as an antimalarial and is used to treat methemoglobinemia and urinary tract infections.

Zanamivir is a clinically used antiviral agent that inhibits neurominidase inhibitor that is inhaled as an aerosol to shorten the duration of influenza infections by preventing neuraminidase from releasing virions from the infected cells (U.S. Natl. Lib. of Med., 2010). Recently, multiple bacterial species have been shown to express bacterial neuraminidases capable of cleaving α2,3-sialic acids (Soon et al., 2006). These neuraminidases have been implicated in biofilm formation, with a P. aeruginosa neuraminidase mutant showing decreased ability to colonize the mouse respiratory tract and decreased biofilm production (Soon et al., 2006). It remains to be seen if zanamivir acts in a similar manner in B. burgdorferi.

### 2.3. Agents Used For Treating Other Disease Conditions

It is interesting to note that several highly active drugs identified in our screen, including verteporfin, oltipraz, pyroglutamic acid, pidolic acid (FIG. 1), and dextrorphan tartrate, act on the glutathione/γ-glutamyl pathway used in mammalian cells involved in protection against intracellular damage from free radicals and peroxides. Glutathione (GSH) is a reducing agent produced in the cytoplasm and transferred to the mitochondria by glutathione-s-transferase (GST), where it protects the mitochondria from ROS damage and functions in amino acid transport (Chiou et al., 2010; Tate et al., 1973). Reduced levels of GSH have been linked to increased sensitivity to ROS damage, resulting in mitochondrial swelling and subsequent damage (Chiou et al., 2010; Anderson, 1998).

Verteporfin (Visudyne), a benzophorphyrin derivative, is a photosensitizing agent currently used to treat macular degeneration that affects the γ-glutamyl pathway (Chiou et al., 2010; U.S. Natl. Lib. of Med., 2015; Novartis; 2013). This intravenous drug is transported in oxygenated blood by lipoproteins, and is activated by laser light treatment allowing for precise chemotherapeutic application (Chiou et al., 2010). Verteporfin is a possible effector of cell membrane permeability through ROS lipid peroxidation (Chiou et al., 2010; Pancewicz et al., 2001). Activated verteporfin has been shown to target the mitochondria, producing reactive oxygen radicals and nitric oxide that damage local endothelium and seal leaky vessels (Chiou et al., 2010; U.S. Natl. Lib. of Med, 2015). Verteporfin depletes GSH levels in HepG2 cells after activation possibly through increased nitric oxide production (Chiou et al., 2010). Oltipraz is an organosulfur compound that belongs to the dithiolethione class. It has been shown to inhibit schistosome and prevent formation of cancer. It activates phase II detoxification enzymes in mammalian cells, which results in the binding of glutathione to electrophilic compounds and subsequent protection against reactive oxygen species (ROS) damage (Townsend and Tew, 2003; Kensler et al., 2003). Pyroglutamic acid (PCA) or pidolic acid (pidolate) or 5-oxoproline is an amino acid derivative that is involved in the γ-glutamyl cycle. PCA is a metabolite of glutathione cycle which is broken down to glutamate and cysteine, which are converted back into glutathione (Anderson, 1998) and is used in humans as dietary supplement and skin moisturizer retainer.

The significant number of highly active drugs that act upon this γ-glutamyl pathway suggests that this pathway is important for persisters and that ROS and peroxide-induced damage are important for killing persisters. We anticipate that inhibition of this pathway could be a good therapeutic target for B. burgdorferi persisters for improved treatment of Lyme disease.

2.4. Active Hits that are Topical Agents or Toxic for Internal Use

Thonzonium bromide, benzododecinium chloride, and butyl chloride were found to have very high activities against stationary phase B. burgdorferi (Table 3, FIG. 1). Thonzonium bromide had even comparable activity to daptomycin against stationary phase B. burgdorferi. However, thonzonium bromide is a cationic detergent and surfactant that is used as a topical agent in combination with other compounds to assist in the penetration of cellular membranes (Siles et al., 2013). Thonzonium bromide has been shown to inhibit vacuolar ATPases in yeast, an enzyme that is closely related to the ATPase found in B. burgdorferi (Hayek et al., 2014; Chan et al., 2012; Fraser C M, et al., 1997). In C. albicans, thonzonium bromide was also shown to inhibit ATPases in isolated vacuoles and cause general cellular toxicity (Hayek et al., 2014; Chan et al., 2012). Thonzonium bromide was also shown to be active against preformed C. albicans biofilms (Siles et al., 2013). Benzododecinium chloride is a C12-substituted alkyl chain derivate of the quarternary ammonium detergent benzalkonium chloride that alters cell membrane permeability and can cause cell lysis through lipid dispersion (Daull et al., 2014; Noecker, 2001). Benzododecinium chloride was shown in S. aureus to have higher activity against the biofilm form of the bacteria than the free planktonic form in vitro (Cabo et al., 2009). Since thonzonium bromide and benzododecinium chloride have strong detergent properties causing generalized cellular damage in humans, they may not be used directly for Lyme treatment. However, the high activity of these drugs against B. burgdorferi persisters suggests both the cell membrane and biofilms are potential targets for future persister drug design.

It is worth noting that the most active hits from the compound library screen are those that affect cell membranes (benzododecinium chloride, thonzonium bromide, zanamivir). This is consistent with our previous finding that daptomycin and clofazimine may act on the cell membrane to show their high activity against B. burgdorferi persisters (Feng J, Wang T, Shi W, Zhang S, Sullivan D, Auwaerter P G, 2014). Indeed, agents that target bacterial cell membranes have been found to be active against persisters in different bacterial pathogens such as M. tuberculosis and E. coli (Zhang et al., 2003; Niu et al., 2015; Hurdle et al., 2011). Other active hits that show good activity against B. burgdorferi persisters interfere with energy production (thonzonium bromide, oxantel) and ROS production (verteporfin, oltipraz, pyroglutamic acid, pidolic acid). Our data showed that these three types of agents, cell membrane disruptors, energy inhibitors and ROS producers, are generally more active against the B. burgdorferi persisters than the more conventional antibiotics inhibit cell wall, protein, RNA, and DNA syntheses (Table 1, Table 3). Future studies are needed to assess the activity of these agents in combination with Lyme antibiotics for more effective eradication of B. burgdorferi persisters in vitro (Feng J, Auwaerter P G, Zhang Y, 2015) and in vivo.

### 3. EXPERIMENTAL SECTION

### 3.1. Strain and Culture Techniques

Borrelia burgdorferi strain B31 (ATCC35210) was received from the American Type Tissue Collection (Manassas, Va., USA) and was grown in BSK-H medium (HiMedia Laboratories, Mumbai, India) and 6% rabbit serum (Sigma Aldrich, St. Louis, Mo., USA). The culture was filtered and sterilized using a 0.2 mm filter and incubated in capped sterile 50 mL conical tubes (BD Biosciences, CA, USA) at 33° C. for 7 days without antibiotics until the culture reached stationary phase. Seven day old stationary phase cultures were transferred to a 96 well culture plate for evaluation of drugs active B. burgdorferi persister.

### 3.2. Microscopy

The cultures were examined using a Nikon Eclipse E800 microscope with differential interference contrast and epifluorescent illumination. The pictures were captured using a SPOT slider color camera. A SYBR Green I/PI assay was used to assess the viability of the bacterial sample using the ratio of live to dead B. burgdorferi (measured with green and red fluorescence, respectively) as measured by a plate reader. The cellular counts were made by capturing three images representative of the bacterial samples using epifluorescence microscopy and quantitatively analyzed using Image Pro-Plus software to calculate the fluorescence intensity as described (Feng J, Wang T, Zhang S, Shi W, Zhang Y, 2014).

3.3. Drug Library Screens for Activity Against B. burgdorferi Persisters In Vitro

The FDA drug library screens against the stationary phase B. burgdorferi persister model were performed as described (Feng J, Wang T, Shi W, Zhang S, Sullivan D, Auwaerter P G, 2014). Briefly, prediluted drug stock (10 μL) was added to 7 day old stationary phase B. burgdorferi culture (90 μL) to achieve a 50 μM final drug concentration per well. The plates were then incubated at 33° C. for 7 days at which point the SYBR/PI rapid viability assay was performed in a fluorescence plate reader to obtain the green-red fluorescence ratio. The top hits from the SYBR/PI assay were then examined using epifluorescence microscopy to ensure accuracy of the SYBR/PI readings and to ensure no fluorescent contamination from colored test drugs as described previously (Feng J, Wang T, Shi W, Zhang S, Sullivan D, Auwaerter P G, 2014).

### 4. CONCLUSIONS

In this study, we present the results of 113 active hits that have higher activity against the stationary phase B. burgdorferi than the currently used Lyme antibiotics. Many antimicrobial agents (antibiotics, antivirals, antifungals, anthelmintics or antiparasitics) used for treating other infections were found to have better activity than the current Lyme antibiotics. These include antibacterials such as rifamycins (3-formal-rifamycin, rifaximin, rifamycin SV), thiostrepton, quinolone drugs (sarafloxacin, clinafloxacin, tosufloxacin), and cell wall inhibitors carbenicillin, tazobactam, aztreonam; antifungal agents such as fluconazole, mepartricin, bifonazole, climbazole, oxiconazole, nystatin; antiviral agents zanamivir, nevirapine, tilorone; antimalarial agents artemisinin, methylene blue, and quidaldine blue; antihelmintic and antiparasitic agents toltrazuril, tartar emetic, potassium antimonyl tartrate trihydrate, oxantel, closantel, hycanthone, pyrimethamine, and tetramisole. Interestingly, drugs used for treating other non-infectious conditions including verteporfin, oltipraz, pyroglutamic acid, pidolic acid, and dextrorphan tartrate, that act on glutathione/γ-glutamyl pathway involved in protection against free radical damage, and also antidepressant drug indatraline, were found to have high activity against stationary phase B. burgdorferi. Among the active hits, agents that affect cell membranes, energy production, and reactive oxygen species production are more active against the B. burgdorferi persisters than the commonly used antibiotics that inhibit macromolecule biosynthesis. The presently disclosed agents can be used for more effective treatment of Lyme disease.

TABLE 3
Active hits that showed better activity against stationary
phase B. burgdorferi than the current Lyme antibiotics^{a}.

| | | Residual viable | Residual viable |
|---|---|---|---|
| | | cells | cells |
| Drugs (50 μM) | Category | (Microscopy)^{b} | (SYBR/PI)^{c} |
| Control | | 93% | 94% |
| Doxycycline | Lyme antibiotic | 75% | 67% |
| Amoxicillin | Lyme antibiotic | 76% | 76% |
| Daptomycin | Antibiotic | 35% | 28% |
| Verteporfin | Ophthalmic | 47% | 27% |
| Thonzonium bromide | Antiseptic | 33% | 31% |
| Tetrachloroethylene | Anthelmintic | 53% | 36% |
| Benzododecinium chloride | Antiseptic | | 40% |
| Butyl chloride (1- | Anthelminthic | | 41% |
| Chlorobutane) | | | |
| 3-formyl Rifamycin | Antibacterial | 59% | 42% |
| | (tuberculostatic) | | |
| Potassium antimonyl tartrate | Anthelmintic | 45% | 42% |
| trihydrate | | | |
| Toltrazuril | Antiprotozoal | | 43% |
| | (Coccidostat) | | |
| Thiostrepton | Antibiotic | 66% | 43% |
| Pyroglutamic acid, DL (DL- | Topical | | 43% |
| 2-Pyrrolidone-5-Carboxylic | Antiseptic | | |
| Acid), Pidolic acid | | | |
| Mepartricin | Antifungal | | 43% |
| Tilorone dihydrochloride | Antiviral | | 44% |
| Oxantel | Anthelmintic | | 44% |
| Hycanthone | Anthelmintic | | 45% |
| | (Schistosoma) | | |
| Pyrimethamine | Antiprotozoal | 55% | 45% |
| | (Toxoplasma) | | |
| Trilocarban (3,4,4′- | Antiseptic | | 45% |
| Trichlorocarbanilide) | | | |
| Carbenicillin | Antibiotic | 64% | 46% |
| Oltipraz | Antitumor | | 46% |
| Bitoscanate | Anthelmintic | | 46% |
| Sarafloxacin HCl | Antibiotic | | 47% |
| Bacitracin zinc salt | Antibiotic | 60% | 47% |
| Dextrorphan tartrate | Analgesic | 43% | 47% |
| Tetramisole | Anthelmintic | | 48% |
| Bifonazole | Antifungal | 50% | 48% |
| Ethacridine lactate | Antiseptic | | 48% |
| Zanamivir | Antiviral | | 49% |
| Aluminum lactate | Antiseptic | | 49% |
| p-Arsanilic acid | Antibacterial | | 49% |
| Artemesinin | Antimalarial | | 49% |
| Nifursol | Antiprotozoal | | 51% |
| Nevirapine | Antiviral | | 51% |
| Rifaximin | Antibiotic | | 51% |
| Oxibendazole | Anthelminthic | | 51% |
| Metrifonate | Anthelmintic | | 51% |
| Indatraline hydrochloride | Monoamine | 43% | 51% |
| | transporter | | |
| | inhibitor | | |
| Florfenicol | Antibiotic | | 53% |
| Benznidazole | Antiprotozoal | | 53% |
| Ganciclovir | Antiviral | | 53% |
| Tazobactam | Antibiotic | 56% | 54% |
| Oxfendazole | Anthelminthic | | 54% |
| Phenothiazine | Anthelminthic | 53% | 54% |
| Flubendazole | Anthelminthic | | 54% |
| Midecamycin | Antibiotic | | 54% |
| Fluconazole | Antifungal | 45% | 55% |
| Docosanol | Antiviral | | 55% |
| | (topical) | | |
| Aztreonam | Antibiotic | 50% | 55% |
| Benzoylpas calcium (4- | Antibiotic | | 55% |
| Benzamido salicylic acid, | | | |
| calcium salt) | | | |
| Trifluridine | Antiviral | | 55% |
| | (ophthalmic) | | |
| Undecylenic acid | Antifungal | 74% | 55% |
| | (topical) | | |
| Closantel | Anthelmintic. | | 56% |
| Cefixime | Antibiotic | 56% | 56% |
| Thiamphenicol | Antibiotic | | 57% |
| Ricobendazole | Anthelmintic | | 57% |
| (Albendazole oxide) | | | |
| Sulfamoxole | Antibiotic | 55% | 57% |
| Clopidol | Antibacterial | | 57% |
| | Coccidiostat | | |
| Tosufloxacin | Antibiotic | | 57% |
| Metampicillin | Antibiotic | | 57% |
| Amikacin | Antibiotic | | 57% |
| Lamivudine | Antiviral | | 58% |
| Cephalosporin C | Antibiotic | | 58% |
| Sulfachlorpyridazine | Antibiotic | | 58% |
| Lomofungin | Antibiotic | | 58% |
| Artesunate | Antimalarial. | | 58% |
| Valacyclovir | Antiviral | | 58% |
| Carzenide (4- | Antibiotic | | 59% |
| Carboxybenzenesulfonamide) | | | |
| Clinafloxacin | Antibiotic | | 60% |
| Efavirenz | Antiviral | | 60% |
| Cefsulodin | Antibiotic | | 60% |
| Cloxyquin (5-chloro-8- | Antibacterial | | 60% |
| hydroxy-quinoline) | | | |
| Symclosene | Antibacterial | | 60% |
| (Trichloroisocyanuric acid) | (topical) | | |
| Didanosine (2′-3′- | Antiviral | | 61% |
| dideoxyinosine) | | | |
| Floxuridine (5- | Antiviral | | 61% |
| fluorodeoxyuridine) | | | |
| Cyacetacide | Antibacterial | | 61% |
| Roxithromycin | Antibiotic | 65% | 62% |
| Oxiconazole nitrate | Antifungal | | 62% |
| Climbazole | Antifungal | 71% | 62% |
| Protionamide | Antibacterial | | 63% |
| | (tuberculostatic) | | |
| Ribavirin | Antiviral | | 63% |
| Griseofulvin | Antifungal | | 63% |
| Rifamycin SV | Antibiotic | 60% | 63% |
| Salicylanilide | Antifungal | | 63% |
| | (topical). | | |
| Diclazuril | Antibacterial | | 63% |
| Imiquimod | Antiviral | | 64% |
| Penciclovir | Antiviral | 60% | 64% |
| Nystatin | Antifungal | | 64% |
| Ampicillin | Antibiotic | | 64% |
| Puromycin | Antibiotic | 48% | 65% |
| Stavudine (2′,3′-Didehydro- | Antiviral | | 65% |
| 3′-Deoxythymidine) | | | |
| Potassium iodide | Antifungal | | 65% |
| Voriconazole | Antifungal | | 65% |
| Penimepicycline | Antibiotic | 60% | 65% |
| Amantadine hydrochloride | Antiviral | | 65% |
| Nitroxoline (8-hydroxy 5- | Antibiotic | | 66% |
| nitroquinoline) | | | |
| 4-Aminosalicylic acid | Antibacterial | | 66% |
| Ciclopirox olamine | Antifungal | | 66% |
| Nelfinavir mesylate | Antiviral | | 66% |
| Anisomycin | Antibiotic | | 68% |
| Betamipron (n-benzoyl-b- | Antibacterial | | 68% |
| alanine) | | | |
| Famciclovir | Antiviral | | 68% |
| Flucytosine (5- | Antifungal | 66% | 68% |
| Fluorocytosine) | | | |
| Clotrimazole | Antifungal | | 68% |
| Rimantadine | Antiviral | | 68% |
| Pazufloxacin | Antibiotic | | 69% |
| Carbadox | Antibacterial | | 69% |
| Amantadine hydrochloride | Antiviral | | 69% |
| Dibekacin | Antibiotic | | 70% |
| Clorsulon | Anthelmintic | | 71% |
| | (Trematodes) | | |
| Thiacetazone (Amithiozone) | Antibacterial | | 73% |
| | (tuberculostatic) | | |
| Fleroxacin | Antibiotic | | 73% |
| Clofoctol | Antibiotic | | 73% |
| Butoconazole nitrate | Antifungal | | 74% |
| | (topical) | | |
| Quinaldine blue | Antimalarial | 35% | Over range^{d} |
| Methylene blue hydrate | Antimethemoglobinemic | 40% | Over range^{d} |

^{a}Stationary phase B. burgdorferi (7-day old) cells were treated with drugs for 7 days.
^{b}Residual viable B. burgdorferi was assayed by epifluorescence microscope counting.
^{c}Residual viable B. burgdorferi was calculated according to the regression equation and ratio of Green/Red fluorescence obtained by SYBR Green I/PI assay.
^{d}The value is higher than the drug free control.

### REFERENCES

All publications, patent applications, patents, and other references mentioned in the specification are indicative of the level of those skilled in the art to which the presently disclosed subject matter pertains. All publications, patent applications, patents, and other references (e.g., websites, databases, etc.) mentioned in the specification are herein incorporated by reference in their entirety to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Although the foregoing subject matter has been described in some detail by way of illustration and example for purposes of clarity of understanding, it will be understood by those skilled in the art that certain changes and modifications can be practiced within the scope of the appended claims.
Anderson M E. Glutathione: an overview of biosynthesis and modulation. Chem Biol Interact. 1998; 111:1-14.
Aucott J N, Rebman A W, Crowder L A, Kortte K B. Post-treatment Lyme disease syndrome symptomatology and the impact on life functioning: is there something there? Qual Life Res. 2013; 22:75.
Barthold S W, Hodzic E, Imai D M, Feng S, Yang X, Luft B J. Ineffectiveness of tigecycline against persistent Borrelia burgdorferi. Antimicrobial Agents and Chemotherapy. 2010; 54(2):643-51. Epub Dec. 10, 2009.
Bayer M E, L. Z, H. B M. Borrelia burgdorferi DNA in the Urine of Treated Patients with Chronic Lyme Disease Symptoms. A PCR Study of 97 Cases. Infection. 1996; 24(5):347.
Cabo M L, Herrera J J, Crespo M D, Pastoriza L. Comparison among the effectiveness of ozone, nisin and benzalkonium chloride for the elimination of planktonic cells and biofilms of Staphylococcus aureus CECT4459 on polypropylene. Food Control. 2009; 20(5):521-5.
Chan C Y, Prudom C, Raines S M, Charkhzarrin S, Melman S D, De Haro L P, et al. Inhibitors of V-ATPase proton transport reveal uncoupling functions of tether linking cytosolic and membrane domain of V0 subunit a (vph1p). J Biol Chem. 2012; 287(13):10236-50.
Chiou J F, Wang Y H, Jou M J, Liu T Z, Shiau C Y. Verteporfin-photoinduced apoptosis in HepG2 cells mediated by reactive oxygen and nitrogen species intermediates. Free Radic Res. 2010; 44(2):155-70.
Daull P, Lallemand F, Garrique J S. Benefits of cetalkonium chloride cationic oil-in-water nanoemulsions for topical opthalmic drug delivery. J Pharm Pharmacol. 2014; 66(4):531-41.
Embers M E, Barthold S W, Borda J T, Bowers L, Doyle L, Hodzic E, et al. Persistence of Borrelia burgdorferi in rhesus macaques following antibiotic treatment of disseminated infection. PLoS One. 2012; 7(1):e29914. Epub Jan. 19, 2012.
Feder H M, Jr., Johnson B J, O'Connell S, Shapiro E D, Steere A C, Wormser G P, et al. A critical appraisal of “chronic Lyme disease”. N Engl J Med. 2007; 357(14):1422-30.
Feng J, Auwaerter P G, Zhang Y. Drug Combinations against Borrelia burgdorferi Persisters In Vitro: Eradication Achieved by Using Daptomycin, Cefoperazone and Doxycycline. PLoS One. 2015; 10(3):e0117207. Epub Mar. 25, 2015.
Feng J, Shi W, Zhang S, Zhang Y. Identification of New Compounds with High Activity against Borrelia burgdorferi Persisters from the NCI Compound Collection. Emerg Microb Infect. 2015; 4, e31; doi:10.1038/emi.2015.31.
Feng J, Wang T, Shi W, Zhang S, Sullivan D, Auwaerter P G, et al. Identification of Novel Activity against Borrelia burgdorferi Persisters Using an FDA Approved Drug Library. Emerg Microb Infect. 2014; Jul. 2, 2014: 3, e49; doi:10.1038/emi.2014.53.
Feng J, Wang T, Zhang S, Shi W, Zhang Y. An Optimized SYBR Green I/PI Assay for Rapid Viability Assessment and Antibiotic Susceptibility Testing for Borrelia burgdorferi. PLoS One. 2014; 9(11):e111809. doi:10.1371/journal.pone.0111809.
Fraser C M, Casjens S, Hang W M, Sutton G G, Clayton R, Lathigra R, et al. Genomic Sequence of a Lyme disease spirochaete, Borrelia burgdorferi. Nature. 1997; 390(6660):580-6.
Hayek S R, Lee S A, Parra K J. Advances in targeting the vacuolar proton-translocating ATPase (V-ATPase) for anti-fungal therapy. Front Pharmacol. 2014; 5(4).
Hinckley A F, Connally N P, Meek J I, Johnson B J, Kemperman M M, Feldman K A, et al. Lyme disease testing by large commercial laboratories in the United States. Clinical infectious diseases: an official publication of the Infectious Diseases Society of America. 2014; 59(5):676-81.
Hodzic E, Feng S, Holden K, Freet K J, Barthold S W. Persistence of Borrelia burgdorferi following antibiotic treatment in mice. Antimicrobial Agents and Chemotherapy. 2008; 52(5):1728-36. Epub Mar. 5, 2008.
Hodzic E, Imai D, Feng S, Barthold S W. Resurgence of Persisting Non-Cultivable Borrelia burgdorferi following Antibiotic Treatment in Mice. PLoS One. 2014; 9(1):e86907. Epub Jan. 28, 2014.
Hurdle J G, O'Neill A J, Chopra I, Lee R E. Targeting bacterial membrane function: an underexploited mechanism for treating persistent infections. Nat Rev Micro. 2011; 9(1):62-75.
Kensler T W, Qian G S, Chen J G, Groopman J D. Translational strategies for cancer prevention in liver. Nat Rev Cancer. 2003; 3(5):321-9.
Lyme Disease. Center for Disease Control and Prevention; 2015 [cited 2015 Jun. 5, 2015]; Available from: http://www.cec.gov/lyme/.
Marques A, Telford S R, 3rd, Turk S P, Chung E, Williams C, Dardick K, et al. Xenodiagnosis to Detect Borrelia burgdorferi Infection: A First-in-Human Study. Clin Infect Dis. 2014; 58(7):937-45. Epub Feb. 14, 2014.
Niu H, Cui P, Shi W, Zhang S, Feng J, Wang Y, et al. Identification of Anti-Persister Activity against Uropathogenic Escherichia coli from a Clinical Drug Library. Antibiotics. 2015; 4:179-87.
Noecker R. Effects of common ophthalmic preservatives on ocular health. Advances in Therapy. 2001; 18(5):205-15.
Pancewicz S A, Skrzdlewska E, Hermanowska-Szpakowicz T, Zajkowska J M, Kondrusik M. Role of reactive oxygen species (ROS) in patients with erythema migrans, an early manifestation of Lyme borreliosis. Med Sci Monit. 2001; 7(6):1230-5.
Phillips S E, Mattman L H, Hulinska D, Moayad H. A Proposal for the Reliable Culture of Borrelia burgdorferi from Patients with Chronic Lyme Disease, Even from Those Previously Aggressively Treated. Infection. 1998; 26:364.
Post Treatment Lyme Disease. CDC; 2015 [cited 2015 Jun. 5]; Available from: http://www.cdc.gov/lyme/postLDS.
Radolf J D, Caimano M J, Stevenson B, Hu L T. Of ticks, mice and men: understanding the dual-host lifestyle of Lyme disease spirochaetes. Nat Rev Microbiol. 2012; 10(2):87-99.
Sapi E, Kaur N, Anyanwu S, Luecke D F, Datar A, Patel S, et al. Evaluation of in-vitro antibiotic susceptibility of different morphological forms of Borrelia burgdorferi. Infect Drug Resistance. 2011; 4:97-113. Epub Jul. 15, 2011.
Siles S A, Srinivasan A, Pierce C G, Lopez-Ribot J L, Ramasubramanian A K. High-Throughput Screening of a Collection of Known Pharmacologically Active Small Compounds for Identification of Candida albicans Biofilm Inhibitors. Antimicrob Agents Chemother. 2013; 57(8):3681-33687.
Soon G, Muir A, Gomez M I, Waks J, Reddy B, Planet P, et al. Bacterial neuraminidase facilitates mucosal infection by participating in biofilm production. J Clin Invest. 2006; 116(8):2297-305.
Straubinger R K, Summers B A, Chang Y F, Appel M J. Persistence of Borrelia burgdorferi in experimentally infected dogs after antibiotic treatment. J Clin Microbiol. 1997; 35(1):111-6. Epub Jan. 1, 1997.
Tate S S, Ross L L, Meister A. The gamma-Glutamyl Cycle in the Choroid Plexus: Its Possible Function in Amino Acid Transport. Proc Natl Acad Sci. 1973; 70(5):1447-9.
Townsend D M, Tew K D. The role of glutathione-S-transferase in anti-cancer drug resistance. Oncogene. 2003; 22:7369-75.
Verteporfin Injection. U.S. National Library of Medicine; 2015 [cited 2015 Jun. 21]; Available from: http://www.nlm.nih.gov/medlineplus/druginfo/meds/a607060.html.
Visudyne: verteporfin. Novartis; 2013; Available from: http://www.novartis.com.au/PI_PDF/vid.pdf.
Wormser G P, Dattwyler R J, Shapiro E D, Halperin J J, Steere A C, Klempner M S, et al. The clinical assessment, treatment, and prevention of lyme disease, human granulocytic anaplasmosis, and babesiosis: clinical practice guidelines by the Infectious Diseases Society of America. Clinical infectious diseases: an official publication of the Infectious Diseases Society of America. 2006; 43(9):1089-134. Epub Oct. 10, 2006.
Zanamivir Oral Inhalation. U.S. National Library of Medicine; 2010 [cited 2015 Jun. 24]; Available from: http://www.nlm.nih.gov/medlineplus/druginfo/meds/a699021.html.
Zhang Y, Wade M M, Scorpio A, Zhang H, Sun Z. Mode of action of pyrazinamide: disruption of Mycobacterium tuberculosis membrane transport and energetics by pyrazinoic acid. J Antimicrob Chemother. 2003; 52(5):790-5.
Zhang Y. Persisters, Persistent Infections and the Yin-Yang Model. Emerg Microb Infect (Nature Publishing Group). 2014; 3, e3; doi:10.1038/emi.2014.3.

1. A method for inhibiting the growth and/or survival of bacteria from the Borrelia genus, the method comprising contacting bacteria from the Borrelia genus with an effective amount of at least one agent that inhibits the glutathione/γ-glutamyl pathway.

2. The method of claim 1, further comprising treating Lyme disease in a patient in need thereof, the method comprising administering to a patient an effective amount of at least one agent that inhibits the glutathione/γ-glutamyl pathway in the patient.

3. The method of claim 1, wherein the at least one agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

4. The method of claim 1, wherein the at least one agent is a cell membrane disruptor, an energy inhibitor, and/or a reactive oxygen species (ROS) producer.

5. The method of claim 1, wherein the at least one agent is selected from the group consisting of verteporfin, oltipraz, pyroglutamic acid, and dextrorphan tartrate.

6. A method for inhibiting the growth and/or survival of bacteria from the Borrelia genus, the method comprising contacting bacteria from the Borrelia genus with an effective amount of at least one agent selected from the group consisting of verteporfin, thonzonium bromide, tetrachloroethylene, benzododecinium chloride, butyl chloride (1-chlorobutane), 3-formyl rifamycin, potassium antimonyl tartrate (tartar emetic), toltrazuril, thiostrepton, pyroglutamic acid, mepartricin, tilorone, oxantel, hycanthone, pyrimethamine, trilocarban (3,4,4′-trichlorocarbanilide), carbenicillin, oltipraz, bitoscanate, sarafloxacin, bacitracin, dextrorphan tartrate, tetramisole, bifonazole, ethacridine lactate, zanamivir, aluminum lactate, p-arsanilic acid, nifursol, nevirapine, rifaximin, oxibendazole, metrifonate, indatraline, florfenicol, benznidazole, ganciclovir, tazobactam, oxfendazole, phenothiazine, flubendazole, midecamycin, fluconazole, docosanol, aztreonam, benzoylpas (4-Benzamido salicylic acid), trifluridine, undecylenic acid, closantel, cefixime, thiamphenicol, ricobendazole (albendazole oxide), sulfamoxole, clopidol, tosufloxacin, metampicillin, amikacin, lamivudine, cephalosporin C, sulfachlorpyridazine, lomofungin, artesunate, valacyclovir, carzenide (4-carboxybenzenesulfonamide), clinafloxacin, efavirenz, cefsulodin, cloxyquin (5-chloro-8-hydroxy-quinoline), symclosene (trichloroisocyanuric acid), didanosine (2′-3′-dideoxyinosine), floxuridine (5-fluorodeoxyuridine), cyacetacide, roxithromycin, oxiconazole nitrate, climbazole, protionamide, ribavirin, griseofulvin, rifamycin SV, salicylanilide, diclazuril, imiquimod, penciclovir, nystatin, ampicillin, puromycin, stavudine (2′,3′-didehydro-3′-deoxythymidine), potassium iodide, voriconazole, penimepicycline, amantadine, nitroxoline (8-hydroxy 5-nitroquinoline), 4-aminosalicylic acid, ciclopirox olamine, nelfinavir mesylate, anisomycin, betamipron (n-benzoyl-b-alanine), famciclovir, flucytosine (5-fluorocytosine), clotrimazole, rimantadine, pazufloxacin, carbadox, amantadine, dibekacin, clorsulon, thiacetazone (amithiozone), fleroxacin, clofoctol, butoconazole nitrate, quinaldine blue, and methylene blue.

7. The method of claim 6, wherein the at least one agent is selected from the group consisting of verteporfin, 3-formyl rifamycin, rifaximin, rifamycin SV, sarafloxacin, clinafloxacin, tosufloxacin, fluconazole, climbazole, tilorone, artemisinin, potassium antimonyl tartrate tryhydrate, tartar emetic, closantel, toltrazuril, thiostrepton, mepartricin, tilorone, oxantel, pyroglutamic acid, hycanthone, pyrimethamine, carbenicillin, oltipraz, bitoscanate, sarafloxacin, bacitracin, dextrorphan tartrate, tetramisole, bifonazole, ethacridine lactate, zanamivir, oxibendazole, indatraline, nevirapine, ganciclovir, phenothiazine, oxfendazole, flubendazole, tazobactam, aztreonam, benzoylpas, fluconazole, cefixime, sulfamoxole, tosufloxacin, lamivudine, cefsulodin, didanosine, floxuridine, cyacetacide, oxiconazole, roxithromycin, ribavirin, griseofulvin, rifamycin SV, penciclovir, nystatin, penimepicycline, puromycin, quinaldine blue, and methylene blue.

8. The method of claim 6, wherein the at least one agent is selected from the group consisting of verteporfin, oltipraz, pyroglutamic acid, dextrorphan tartrate, zanamivir, 3-formyl rifamycin, quinaldine blue, and methylene blue.

9. A method for treating Lyme disease in a patient in need thereof, the method comprising administering to a patient an effective amount of at least one agent selected from the group consisting of verteporfin, thonzonium bromide, tetrachloroethylene, benzododecinium chloride, butyl chloride (1-chlorobutane), 3-formyl rifamycin, potassium antimonyl tartrate (tartar emetic), toltrazuril, thiostrepton, pyroglutamic acid, mepartricin, tilorone, oxantel, hycanthone, pyrimethamine, trilocarban (3,4,4′-trichlorocarbanilide), carbenicillin, oltipraz, bitoscanate, sarafloxacin, bacitracin, dextrorphan tartrate, tetramisole, bifonazole, ethacridine lactate, zanamivir, aluminum lactate, p-arsanilic acid, nifursol, nevirapine, rifaximin, oxibendazole, metrifonate, indatraline, florfenicol, benznidazole, ganciclovir, tazobactam, oxfendazole, phenothiazine, flubendazole, midecamycin, fluconazole, docosanol, aztreonam, benzoylpas (4-Benzamido salicylic acid), trifluridine, undecylenic acid, closantel, cefixime, thiamphenicol, ricobendazole (albendazole oxide), sulfamoxole, clopidol, tosufloxacin, metampicillin, amikacin, lamivudine, cephalosporin C, sulfachlorpyridazine, lomofungin, artesunate, valacyclovir, carzenide (4-carboxybenzenesulfonamide), clinafloxacin, efavirenz, cefsulodin, cloxyquin (5-chloro-8-hydroxy-quinoline), symclosene (trichloroisocyanuric acid), didanosine (2′-3′-dideoxyinosine), floxuridine (5-fluorodeoxyuridine), cyacetacide, roxithromycin, oxiconazole nitrate, climbazole, protionamide, ribavirin, griseofulvin, rifamycin SV, salicylanilide, diclazuril, imiquimod, penciclovir, nystatin, ampicillin, puromycin, stavudine (2′,3′-didehydro-3′-deoxythymidine), potassium iodide, voriconazole, penimepicycline, amantadine, nitroxoline (8-hydroxy 5-nitroquinoline), 4-aminosalicylic acid, ciclopirox olamine, nelfinavir mesylate, anisomycin, betamipron (n-benzoyl-b-alanine), famciclovir, flucytosine (5-fluorocytosine), clotrimazole, rimantadine, pazufloxacin, carbadox, amantadine, dibekacin, clorsulon, thiacetazone (amithiozone), fleroxacin, clofoctol, butoconazole nitrate, quinaldine blue, and methylene blue.

10. The method of claim 9, wherein the at least one agent is selected from the group consisting of verteporfin, 3-formyl rifamycin, rifaximin, rifamycin SV, sarafloxacin, clinafloxacin, tosufloxacin, fluconazole, climbazole, tilorone, artemisinin, potassium antimonyl tartrate tryhydrate, tartar emetic, closantel, toltrazuril, thiostrepton, mepartricin, tilorone, oxantel, pyroglutamic acid, hycanthone, pyrimethamine, carbenicillin, oltipraz, bitoscanate, sarafloxacin, bacitracin, dextrorphan tartrate, tetramisole, bifonazole, ethacridine lactate, zanamivir, oxibendazole, indatraline, nevirapine, ganciclovir, phenothiazine, oxfendazole, flubendazole, tazobactam, aztreonam, benzoylpas, fluconazole, cefixime, sulfamoxole, tosufloxacin, lamivudine, cefsulodin, didanosine, floxuridine, cyacetacide, oxiconazole, roxithromycin, ribavirin, griseofulvin, rifamycin SV, penciclovir, nystatin, penimepicycline, puromycin, quinaldine blue, and methylene blue.

11. The method of claim 9, wherein the at least one agent is selected from the group consisting of verteporfin, oltipraz, pyroglutamic acid, dextrorphan tartrate, zanamivir, butyl chloride, 3-formyl rifamycin, quinaldine blue, and methylene blue.

12. The method of claim 1, wherein the bacteria is contacted with or the patient is administered the at least one agent and at least one other agent selected from the group consisting of doxycycline, amoxicillin, cefuroxime, ceftriaxone, metronidazole, tinidazole, erythromycin, azithromycin, clarithromycin, penicillin G, cefotaxime, a nonsteroidal anti-inflammatory agent, a corticosteroid, and a disease-modifying antirheumatic drug (DMARD).

13. The method of claim 1, wherein the bacteria are Borrelia burgdorferi.

14. The method of claim 13, wherein the bacteria comprise replicating forms of Borrelia burgdorferi, non-replicating persister forms of Borrelia burgdorferi, and combinations of replicating forms of Borrelia burgdorferi and non-replicating persister forms of Borrelia burgdorferi.

15. The method of claim 13, wherein the bacteria comprise a morphological form of Borrelia burgdorferi selected from the group consisting of round bodies, planktonic, and biofilm.

16. The method of claim 1, wherein the contacting occurs in vitro or in vivo.

17. The method of claim 1, wherein the patient has, or is suspected of having, post-treatment Lyme disease syndrome (PTLDS) and/or antibiotic refractory Lyme arthritis.

18. The method of claim 1, wherein the at least one agent inhibits the growth and/or survival of greater than about 35 percent of the population of non-replicating persister forms of bacteria.

19. The method of claim 18, wherein the at least one agent inhibits the growth and/or survival of greater than about 50 percent of the population of non-replicating persister forms of bacteria.

20. The method of claim 2, wherein the patient is a human.
